(19) United States
(12) Plant Patent Application Publication
(10) Pub. No.: US 2011/0197324 P1
(43) Pub. Date: Aug. 11, 2011

(54) Hybrid tea rose plant named 'PC1249'

(50) ;

(75) Inventor: Juan Requena Ruiz, Cordoba (ES)

(73) Assignee: Plantas Continental, S.A., Cordoba (ES)

(21) Appl. No.: 13/066,218

(22) Filed: Apr. 8, 2011

(51) Int. Cl. A01H 5/00

(52) U.S. Cl. PLT/138

(57) ABSTRACT

A new variety of Hybrid Tea Rose plant named ‘PC1249’, particularly characterized by hot pink flowers, high productivity, large stems, and good winter hardiness, is disclosed.

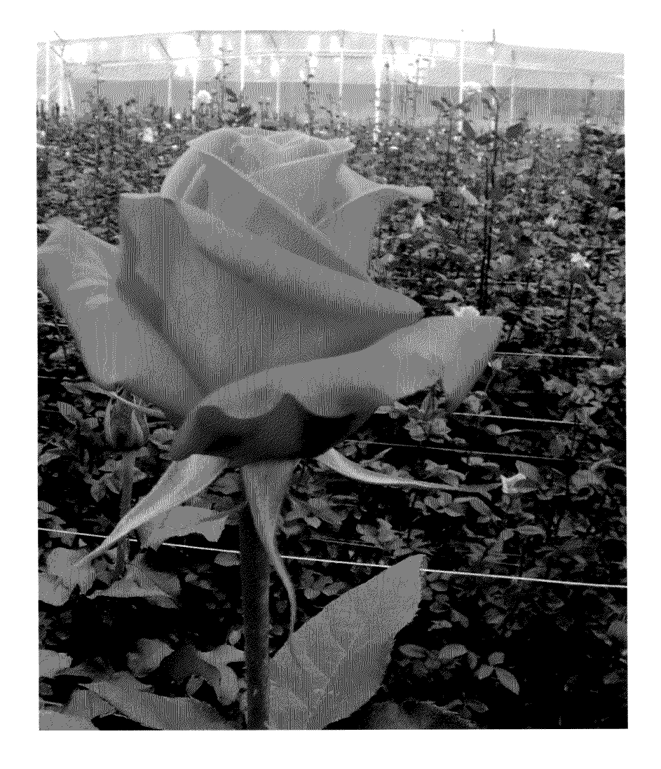

### GENUS AND SPECIES

Rosa hybrida

### VARIETY DENOMINATION

‘PC1249’

### BACKGROUND OF THE NEW PLANT

The present invention comprises a new and distinct variety of Hybrid Tea Rose plant, botanically known as Rosa hybrida, and hereinafter referred to by the variety name ‘PC1249’. ‘PC1249’ was discovered as a seedling resulting from a controlled cross in April, 2001 in Córdoba, Spain between the proprietary Hybrid Tea Rose female parent, ‘1200’ (unpatented), and the proprietary Hybrid Tea Rose male parent, ‘4900’ (unpatented). A single plant selection was subsequently chosen for further evaluation and for asexual propagation.

The new variety ‘PC1249’ was first propagated via vegetative cuttings in April, 2003, in Córdoba, Spain and has been asexually reproduced repeatedly by vegetative cuttings in Córdoba, Spain for approximately two generations. ‘PC1249’ has been found to retain its distinctive characteristics through successive asexual propagations via vegetative cuttings.

Plant Breeder's Rights for this variety were applied for in the following countries: Europe on Jul. 23, 2007, Ecuador on May 29, 2007, and Colombia on Jun. 1, 2009. ‘PC1249’ has not been made publicly available or sold more than one year prior to the filing date of this application.

### SUMMARY OF THE INVENTION

The following are the most outstanding and distinguishing characteristics of this new variety when grown under normal commercial practices in a greenhouse in Córdoba, Spain.

1. Hot pink flowers;

2. Large stems;

3. High productivity;

4. Vigorous growth; and

5. Good winter hardiness.

### DESCRIPTION OF THE PHOTOGRAPH

This new Hybrid Tea Rose plant is illustrated by the accompanying photograph which shows the bloom and foliage of the plant. The colors shown are as true as can be reasonably obtained by conventional photographic procedures. The photograph, which was taken in 2003, shows the flower of a two year old plant grown from rooted cuttings in a greenhouse in Córdoba, Spain under conditions which approximate those generally used in horticultural practice.

### DESCRIPTION OF THE NEW VARIETY

The following detailed descriptions set forth the distinctive characteristics of ‘PC1249’. The data which define these characteristics were collected from asexual reproductions carried out in Córdoba, Spain. The plant history was taken on 2 year old plants grown in a greenhouse in Córdoba, Spain. Color designations are from the Pantone Color Chart dated Apr. 1, 2011 edition, except for the flower petal colors, which are from the Pantone Chart dated Mar. 31, 2008.

### DETAILED BOTANICAL DESCRIPTION OF THE NEW PLANT

Classification:
   Family.—Rosaceae.
   Species.—Rosa hybrida.
   Common name.—Hybrid Tea Rose.
   Denomination.—‘PC1249’.
Plant:
   Form/shape of plant.—Shrub.
   Plant habit.—Generally Erect.
   Growth/vigor.—Vigorous.
   Age at maturity.—2 years.
   Mature plant size.—Height: 2.5 m Width: 0.8 m.
   Lastingness of the blooms on the plant.—18 days.
   Lastingness as a cut flower.—15 days.
   Rootstock.—Natal Briar.
   Winter hardiness.—Good.
Foliage:
   Average number of leaves per plant.—High — approximately 114.
   Size.—Length: 200 mm Width: 150 mm.
   Color, immature leaves.—Upper surface: Reddish Green Group, Pantone 7495C+7646C Lower surface: Reddish Green Group, Pantone 5883C+687C.
   Color, mature leaves.—Upper surface: Deep Dark Green Group, Pantone 5743C Lower surface: Deep Green Light Group, Pantone 5763C and 5773C.
Leaflets:
   Average number of leaflets per leaf.—3 to 7.
   Size.—Length: 100 mm Width: 60 mm.
   Shape.—Elliptical.
   Apex.—Acuminate.
   Base.—Rounded.
   Margin.—Serrated.
   Texture.—Medium.
   Serration type.—Serrated.
   Petiole.—Color: Greenish Yellow Group, Pantone 0131U Length: 30 to 55 mm Diameter: 2 to 3 mm Petiole rachis: Color, upper surface: Reddish Green Group, Pantone 5773C+690C Color, lower surface: Light Green Group, Pantone 7994C Length: 160 to 170 mm Texture: Fibrous Auricles: Present from the middle region narrowing downward, margin comb-shaped Stipules: Length: 20 mm Texture and margin: Fine and serrated Vein: Color, upper surface: Reddish Group, Pantone 682C Color, lower surface: Green Group, Pantone 5773C Pattern: Penninervia.
Wood:
   New wood.—Color: Green Group, Pantone 5753C Bark texture: Smooth Length: 700 mm Diameter: 7.0 to 10.0 mm Internode length: 50 to 60 mm.
   Old wood.—Color: Light Green Group, Pantone 5763C Bark texture: Smooth Length: 1200 mm Diameter: 15.0 mm Internode length: 45 to 55 mm.
Large prickles:
   Quantity on main canes from base per stem.—26 per 100 mm of cane.
   Quantity on laterals from main canes per stem.—9 per 100 mm of cane.
   Form.—Concave.
   Color, immature.—Reddish Green Group, Pantone 5783C+7647C.
   Color, mature.—Brown Group, Pantone 7518C.
Small prickles:
   Quantity on main canes.—21 per 100 mm of cane.
   Quantity on laterals of main canes.—24 per 100 mm of lateral.
   Color, immature.—Light Green Group, Pantone 381C.
   Color, mature.—Reddish Green Group, Pantone 578C+7647C.
Peduncle:
   Length.—136 mm.
   Diameter.—6.0 to 9.0 mm.
   Surface texture.—Prickles present.
   Color.—Light Green Group, Pantone 575C.
   Strength.—Consistent.
Flower bud:
   Size.—Length: 20 mm Width: 15 mm.
   Color, when sepals first divide.—Pink Group, Pantone 7423C.
   Color, when half-blown.—Reddish Group, Pantone 7426C.
Flower bloom:
   Size (average diameter when open).—140 mm.
   Borne.—Singly.
   Form (when first open).—Imbricate.
   Variegations.—Absent.
   Discoloration.—None.
   Fragrance.—Absent.
Sepals:
   Quantity.—5.
   Color.—Upper surface: Grayish Green Group, Pantone 5783C-5793C Lower surface: Reddish Green Group, Pantone 7496C with 7652C.
   Length.—45 mm.
   Width.—10.0 to 12.0 mm.
   Shape.—Weak.
   Surface texture.—Upper surface: Velvety Lower surface: Smooth.
Petals:
   Petalage (number of petals per flower).—33.
   Color, upper surface.—Face petal: Deep Hot Pink Group, Pantone (2008) 1925U.
   Color, lower surface.—Underside of petal: Deep Hot Pink Group, Pantone (2008) 213U.
   Texture (of both surfaces).—Smooth.
   Shape.—Rounded.
   Apex.—Absent.
   Base.—White.
   Margin.—Homogenous.
   Length.—90 mm.
   Width.—80 mm.
   Petaloids.—Absent.
   Persistence.—Good.
Receptacle:
   Color.—Green Group, Pantone 377C.
   Shape.—Pear-shaped.
   Size.—Length: 30.0 mm Width: 15.0 mm.
   Surface texture.—Smooth.
Reproductive parts:
   Stamens.—Number per flower: 200 Length: 5.0 to 10.0 mm Anthers: Length: 3.0 to 5.0 mm Color: Yellow-Orange Group, Pantone 120C Pollen: Color: Light Yellow Group, Pantone 600C Amount: Abundant Filaments: Color: Pinkish Group, Pantone 203C Length: 5.0 to 10.0 mm.
   Pistils.—Number per flower: 100 Length: 4.0 to 6.0 mm Styles: Color: White Length: 9.0 mm Stigmas: Color: Very Light Yellow Group, Pantone 600C Length: 0.5 to 1.0 mm.
   Hips.—None observed.
Seed set: None observed
Disease and insect resistance: The disease and insect resistance of this variety is typical of hybrid tea rose varieties.

### COMPARISON WITH PARENTAL AND COMMERCIAL VARIETIES

‘PC1249’ differs from the female parent, the proprietary Hybrid Tea Rose plant ‘1200’, in that ‘PC1249’ has hot pink flowers, while ‘1200’ has white flowers. Additionally, ‘PC1249’ differs from ‘1200’ in that ‘PC1249’ has large stems of about 70 to 100 cm in diameter, while ‘1200’ has medium stems of about 40 to 60 cm in diameter. Further, ‘PC1249’ is about 30% more productive than ‘1200’.

‘PC1249’ differs from the proprietary male parent, ‘4900’, in that ‘PC1249’ has hot pink flowers, while ‘4900’ has red flowers. In addition, ‘PC1249’ is about 40% more productive than ‘4900’.

‘PC1249’ differs from the commercial comparison variety ‘Hot Princess’ (unpatented), in that ‘PC1249’ has larger flowers than ‘Hot Princess’. In addition, ‘PC1249’ has a larger stem size than ‘Hot Princess’. Further, ‘PC1249’ is more productive and less susceptible to diseases than ‘Hot Princess’.

We claim:

1. A new and distinct variety of Hybrid Tea Rose plant named ‘PC1249’ as shown and described herein.
